(19) United States
(12) Patent Application Publication
(10) Pub. No.: US 2009/0281509 A1
(43) Pub. Date: Nov. 12, 2009

(54) APPARATUS TO COLLECT BODY FLUIDS FOLLOWING LIPOSUCTION SURGERY

(76) Inventor: Michael B. Gellis, Bloomfield Hills, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/118,780

(22) Filed: May 12, 2008

(51) Int. Cl. A61M 1/00

(52) U.S. Cl. 604/316

(57) ABSTRACT

An apparatus to remove and collect body fluids from a skin surgical incision following liposuction surgery. The apparatus includes a compressible suction bulb having an inlet. A patch having an adhesive on one side is attached to the outer epidermal surface by an adhesive so that the patch overlies the incision. The patch includes a central opening and a fluid tube fluidly connects the central opening of the patch to the suction bulb. Consequently, fluid flowing through the skin incision flows through the tube and into the suction bulb for collection and subsequent disposal.

### BACKGROUND OF THE INVENTION

I. Field of the Invention

The present invention relates generally to medical devices and, more particularly, to a device for collecting draining body fluids following liposuction surgery.

II. Description of Material Art

Liposuction surgery is a commonly performed surgery for removing fatty tissues from the interior of the human body. Liposuction surgery may be performed in many areas of the human body including buttocks, legs, abdomen, neck, etc.

In performing liposuction surgery, the surgeon makes a small incision through the skin and then injects a saline solution into the body to loosen the fatty deposits. A suction device then removes the loosened fat from the interior of the patient. Oftentimes, a plurality of different incisions in different areas of the body are performed during a single liposuction operation.

Following the liposuction surgery, a significant amount of the saline solution injected into the body to perform the liposuction surgery remains within the interior of the body. Consequently, rather than tightly closing the incisions made through the skin by the surgeon during the liposuction surgery, the surgeon typically closes these incisions so that the fluid contained within the body is able to drain out of the human body through the surgical incisions during postoperative recovery. This drainage typically ceases after two days.

Although the drainage through the loosely closed surgical incisions following the liposuction surgery is effective in allowing the excess saline solution to evacuate from the body, the ooze of the saline solution through the surgical incisions, together with intermixed blood and any other debris, results in an extremely messy situation since the effluent from the body oftentimes stains clothing, bedding, furniture, and the like. Such staining can, and oftentimes does, result even if the surgical wounds are bandaged following the liposuction surgery.

There is also an unconfirmed concern that body viruses such as HIV and hepatitis A, B or C may also drain out mixed with the body fluids also contaminating clothing and furniture as mentioned above.

### SUMMARY OF THE PRESENT INVENTION

The present invention provides a sterile device which overcomes the above-mentioned disadvantages of liposuction surgery.

In brief the device of the present invention comprises a manually compressible suction bulb having an inlet. The suction bulb is preferably constructed of a plastic material so that, upon compression and the subsequent release of the suction bulb, the suction bulb creates a suction at its inlet as the suction bulb attempts to return to its original uncompressed shape.

A collecting patch has an adhesive on one side and this patch is dimensioned to overlie the surgical incision. In doing so the adhesive secures the patch to the outer skin or epidermal surface surrounding the incision. The patch also includes an opening in its central region which is open to the incision.

A fluid conduit, such as a plastic tube, then fluidly connects the suction bulb inlet to the patch opening. Thus, with the patch properly sealingly attached to the outer skin surface of the patient so that the patch surrounds the incision, the suction from the suction bulb is fluidly connected by the tube to the patch opening. Consequently, fluid flow through the incision passes through the patch opening, then through the tube and finally into the suction bulb. Thereafter, the suction bulb may be emptied as required and recompressed while attached to the tube until no longer necessary.

Alternatively, a plurality of fluid conduits, each having their own patch, may be fluidly coupled to the suction bulb. Such a device would therefore accommodate a liposuction surgery having multiple incisions so that each incision is fluidly connected to and drains fluid into the suction bulb.

### BRIEF DESCRIPTION OF THE DRAWING

A better understanding of the present invention will be had upon reference to the following detailed description when read in conjunction with the accompanying drawing, wherein like reference characters refer to like parts throughout the several views, and in which:

FIG. 1 is a perspective view illustrating a preferred embodiment of the present invention;

FIG. 2 is a view of the suction bulb in a collapsed position;

FIG. 3 is a perspective view illustrating a portion of the present invention; and

FIG. 4 is a view similar to FIG. 1 but illustrating a modification thereof.

### DETAILED DESCRIPTION OF PREFERRED EMBODIMENTS OF THE PRESENT INVENTION

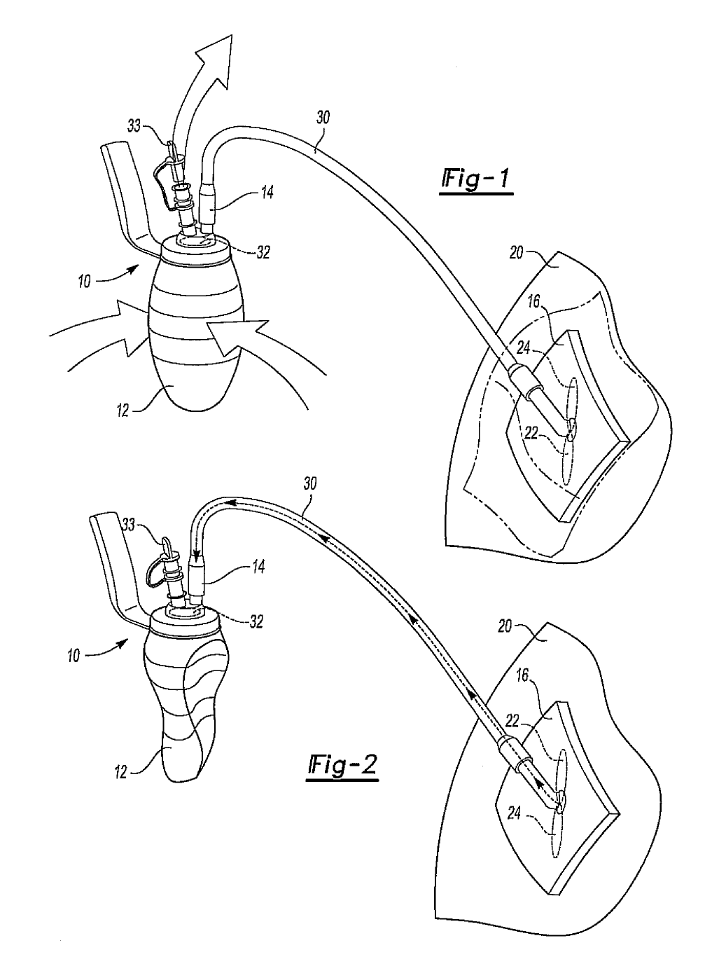

With reference first to FIG. 1, a preferred embodiment of the apparatus 10 of the present invention for removing body fluids from a patient following liposuction surgery is shown. The apparatus includes a suction bulb 12 having an inlet 14. The suction bulb 12 is made of a compressible or collapsible material, such as plastic, so that, upon compression of the suction bulb 12 as shown in FIG. 2, air is expelled out through the inlet 14. Conversely, upon subsequent release of the suction bulb 12, the suction bulb attempts to return to its original shape illustrated in FIG. 1 thus creating a suction at the inlet 14.

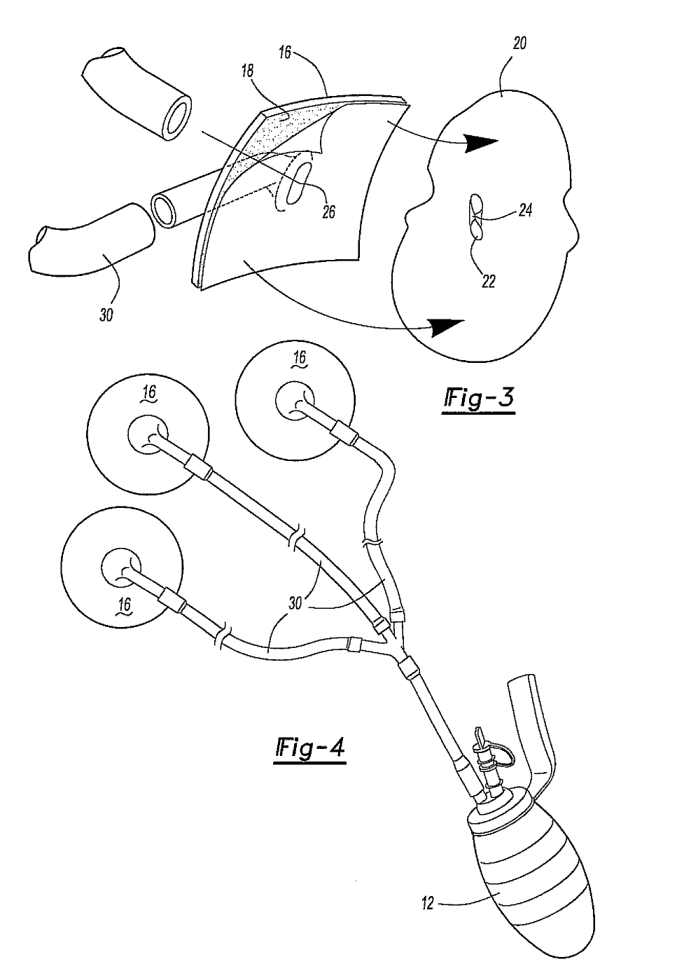

With reference now particularly to FIGS. 3 and 4, a patch 16 includes an adhesive 18 for attaching the patch 16 to an outer epidermal or skin surface 20 of a patient following liposuction surgery. As such, the patient includes one or more incisions 22 which are only partially closed, e.g. by a single stitch 24, so that body fluids can still flow through the incision 22.

The patch 16 is dimensioned to overlie and cover the entire incision 22. However, the patch 16 includes a central opening 26, i.e. an opening spaced inwardly from an outer periphery 28 of the patch 16. This opening 26 is aligned with the incision 22 once the patch 16 is attached to the outer skin or epidermal surface 20. Additionally, the adhesive 18 is selected to provide a fluid-tight seal between the patch 16 and the outer skin surface 20.

With reference to FIGS. 1 and 3, an elongated fluid conduit, such as a flexible plastic tube 30, fluidly sealingly connects the suction bulb inlet 14 with the patch opening 26. Consequently, with a suction bulb 12 in a collapsed position (FIG. 2) and the patch 16 attached to the outer skin surface 20 around the incision 22 following the liposuction surgery, the suction bulb 12 creates a suction at the incision 22. Consequently, fluids flowing through the incision flow through the patch opening 26, tube 30 and into the interior of the suction bulb 12 for collection and subsequent disposal.

With reference again to FIG. 1, a one-way valve 32 is optionally provided fluidly in series with the tube 30 and the interior of the suction bulb 12. This one-way valve 32 permits fluid flow only from the tube and into the suction bulb 12. Furthermore, a selectively closable vent 33 is also provided when the valve 32 is present. This vent 33 is opened to enable the suction bulb to be squeezed and collapsed (FIG. 2), and then closed to trap the suction in the suction bulb.

With reference now to FIG. 4, multiple tubes 30, each having its own associated patch 16, may all be fluidly connected to the suction bulb 12. In this fashion, a single suction bulb 12 is able to collect fluids through multiple incisions following the liposuction surgery.

From the foregoing, it can be seen that the present invention provides a simple yet effective means for collecting fluids through incisions following liposuction surgery which avoids the disadvantages of the previously known practices. Having described my invention, however, many modifications thereto will become apparent to those skilled in the art to which it pertains without deviation from the spirit of the invention as defined by the scope of the appended claims.

I claim:

1. Apparatus to remove and collect body fluids from an epidermal surgical incision following liposuction surgery comprising:
a fluid container,
a patch having an adhesive on one side, said patch dimensioned to overlie the incision so that said adhesive secures said patch to an outer epidermal surface surrounding the incision, said patch having an opening spaced inwardly from an outer periphery of said patch and open to the incision, and
a fluid conduit fluidly connecting said fluid container to said patch opening so that fluid flow through the incision flows through said fluid conduit and into said fluid container.

2. The apparatus as defined in claim 1 and comprising means for creating a suction in said fluid conduit.

3. The apparatus as defined in claim 1 wherein said container comprises a collapsible container with shape memory so that, once collapsed, said container attempts to return to its uncollapsed shape, said fluid conduit being fluidly sealed to said container and open only to an interior of said container.

4. The apparatus as defined in claim 3 and comprising a one-way valve fluidly in series with said container and said fluid conduit so that said valve permits fluid flow only from said patch and to the interior of said container.

5. The apparatus as defined in claim 1 wherein said fluid conduit comprises a tube.

6. The apparatus as defined in claim 1 wherein said container comprises a compressible suction bulb.

7. The apparatus as defined in claim 1 and comprising a plurality of fluid conduits fluidly connected to said container, each fluid conduit having its own patch secured to its free end.

8. Apparatus to remove and collect body fluids from an epidermal surgical incision following liposuction surgery comprising:
a compressible suction bulb having an inlet,
a patch having an adhesive on one side, said patch dimensioned to overlie the incision so that said adhesive secures said patch to an outer epidermal surface surrounding the incision, said patch having an opening spaced inwardly from an outer periphery of said patch and open to the incision, and
a fluid conduit fluidly connecting said suction bulb inlet to said patch opening so that fluid flow through the incision flows through said fluid conduit and into said suction bulb.

9. The apparatus as defined in claim 8 wherein said fluid conduit comprises a flexible tube, said tube being fluidly sealingly connected to said suction bulb inlet.

10. The apparatus as defined in claim 8 wherein said suction bulb is made of a plastic material.

11. The apparatus as defined in claim 8 and comprising a one-way valve fluidly connected in series with said conduit and said suction bulb.

12. The apparatus as defined in claim 11 wherein said valve is contained in said suction bulb adjacent said inlet.
